(19) United States
(12) Patent Application Publication
(10) Pub. No.: US 2005/0266028 A1
(43) Pub. Date: Dec. 1, 2005

(54) Compositions and methods for modulating an immune response

(75) Inventors: David Hurley, Athens, GA (US); Russ Carlson, Athens, GA (US); James Moore, Watkinsville, GA (US); Peter Albersheim, Athens, GA (US)

Correspondence Address: RUDEN, MCCLOSKY, SMITH, SCHUSTER & RUSSELL, P.A., 222 LAKEVIEW AVE, SUITE 800, WEST PALM BEACH, FL, 33401-6112, US

(73) Assignee: University of Georgia Research Foundation, Inc.

(21) Appl. No.: 11/136,963

(22) Filed: May 25, 2005

Related U.S. Application Data
(60) Provisional application No. 60/574,288, filed on May 25, 2004.

(51) Int. Cl. A61K 31/739; A61K 39/02

(52) U.S. Cl. 424/234.1; 514/54

(57) ABSTRACT

Composition and methods relating to a lipopolysaccharide isolated from Rhizobium galegae that enhances an immune response against an antigen yet does not induce a significant inflammatory response are described herein.

### CROSS-REFERENCE TO RELATED APPLICATIONS

The present application claims the priority of U.S. provisional patent application Ser. No. 60/574,288 filed on May. 25, 2004.

### STATEMENT AS TO FEDERALLY SPONSORED RESEARCH

The invention was made with United States government support under grant number GM89585 awarded by the National Institutes of Health and grant number 99-35204-7790 awarded by the United States Department of Agriculture. The United States government may have certain rights in the invention.

### FIELD OF THE INVENTION

The invention relates generally to the fields of immunology and microbiology. More particularly, the invention relates to methods and compositions for enhancing an antigen-specific immune response using at least a portion of a lipopolysaccharide (LPS) derived from Rhizobium galegae.

### BACKGROUND

Adjuvants are agents that non-specifically augment an animal's antigen-specific immune response when mixed with the antigen prior to administration or when administered separately from the antigen. A number of different adjuvants are known. These can be classified into six groups: oil adjuvants, mineral salts, synthetic polymers, lipid vesicles, natural substances, and others. A well-known oil adjuvant is Freund's adjuvant, which is a mineral oil-based adjuvant that also contains an emulsifying agent and, optionally, heat-killed Mycobacterium tuberculosis. Mineral compounds having an adjuvant effect include aluminum sulfate, aluminum hydroxide, aluminum phosphate, and calcium phosphate-based agents. Aluminum compounds are the most commonly used adjuvants in human vaccines as they have exhibited an excellent safety record. Examples of synthetic polymers include synthetic polyribonucleotides (e.g., polyIC and poly IU) and non-ionic polymer surfactants (e.g., pluronic polyol compounds). Natural substances with an adjuvant effect include substances produced by fungi, parasites, and particularly bacteria. The latter include products derived from M. tuberculosis, Bordetella pertussis, Salmonella typhimurium, and Brucellae group bacteria.

LPS is one of the components from enteric bacteria that has been shown to exert a potent adjuvant effect. Because it can also induce a severe local and systemic inflammatory reaction, it has found little use in human or veterinary applications.

### SUMMARY

The invention relates to the discovery that an LPS isolated from the non-enteric bacterium, Rhizobium galegae, and the lipid A portion thereof both can augment an antigen-specific immune response without concomitantly inducing a significant inflammatory response.

Accordingly, the invention features an adjuvant composition that includes an excipient and a purified R. galegae LPS or a purified lipid A portion of an R. galegae LPS. The invention also includes a vaccine composition that includes an adjuvant including a purified R. galegae LPS and/or a purified lipid A from R. galegae LPS, and, optionally, an excipient; and an antigen. The excipient can include an oil which may be a metabolizable oil (e.g., olive oil). It can also include a vitamin such as one or more of vitamin A, vitamin D3, and vitamin E.

Also within the invention is a method of modulating an immune response (e.g., serum antibody production and/or mucosal antibody production) in an animal. This method includes a step of administering to the animal an effective amount of a vaccine composition that includes an adjuvant including a purified R. galegae LPS and/or a purified lipid A from R. galegae LPS, and, optionally, an excipient; and an antigen.

Unless otherwise defined, all technical terms used herein have the same meaning as commonly understood by one of ordinary skill in the art to which this invention belongs. With respect to a molecule such as an LPS molecule, the term “purified” means separated from components that naturally accompany such molecules. Typically, an LPS is purified when it is at least 30% (e.g., 40%, 50%, 60%, 70%, 80%, 90%, and 100%), by weight, free from the lipids, proteins, or other naturally-occurring organic molecules with which it is naturally associated. By the phrase “metabolizable oil” is meant any oil that can be metabolized by an animal.

Although methods and materials similar or equivalent to those described herein can be used in the practice or testing of the present invention, suitable methods and materials are described below. All publications mentioned herein are incorporated by reference in their entirety. In the case of conflict, the present specification, including definitions will control. In addition, the particular embodiments discussed below are illustrative only and not intended to be limiting.

### DETAILED DESCRIPTION

The invention encompasses compositions and methods relating to the use of R. galegae LPS and/or the lipid A portion thereof to enhance an antigen-specific immune response without inducing a significant inflammatory response. R. galegae LPS was purified from in vitro cultures of the rhizobium. Its structure was analyzed and determined to differ from LPSs from enteric bacteria. The intact LPS was separated into carbohydrate (CHO) and lipid A portions.

The effect of R. galegae LPS and the lipid A portion thereof was analyzed in various biological assays. In in vitro assays, the intact LPS, as well as the lipid A portion, were shown to activate complement (C′) more potently than LPS from enteric bacteria. It did not induce TNF-α a production from monocytes in in vitro assays. In bovine cells, R. galegae LPS induced increased radical oxygen (but not nitrogen) production, B cell proliferation, increased CD25 expression, and increased particle uptake by phagocytes. In rabbits and mice, a vaccine preparation including the LPS and an antigen emulsified in an excipient (olive oil and vitamins A, D3, and E) induced primary and secondary serum antibody responses comparable to or better than that induced by a commercial adjuvant, but did not cause fever, injection-site reactions, or any other signs of inflammation. It also induced a stronger mucosal (fecal) antibody response compared to a commercial adjuvant. The number of lymph nodes draining the injection sites that were large enough to recover was fewer in animals receiving R. galegae LPS compared to animals receiving LPS from enteric bacteria.

The below described preferred embodiments illustrate adaptation of these compositions and methods. Nonetheless, from the description of these embodiments, other aspects of the invention can be made and/or practiced based on the description provided below.

### Chemical, Immunological, and Biological Methods

Methods involving conventional chemistry, biochemistry, molecular biology, immunology, and pharmacology techniques are described herein. Such techniques are generally known in the art and are described in detail in methodology treatises such as Current Protocols in Immunology, ed. Coligan et al., John Wiley & Sons, New York; Remington's Pharmaceutical Sciences, 18^{th} Edition, Mack Publishing Company, Easton, Pa., 1990; Methods in Cellular Immunology, 2^{nd} ed., by Rafael Fernandez-Botran and Vaclav Vetricka, CRC Press, Boca Raton, Fla., 2001; Methods of Immunological Analysis, ed. Masseyeff et al., John Wiley & Sons, New York, 1992; and Molecular Cloning: A Laboratory Manual, 3^{rd} ed., vol. 1-3, ed. Sambrook et al., Cold Spring Harbor; Laboratory Press, Cold Spring Harbor, N.Y., 2001.

### LPS

LPS may be isolated from R. galegae by any suitable method. For example, R. galegae may be grown in tryptone/yeast extract medium supplemented with Ca²⁺ in a fermentation apparatus according to the method described in Carlson et al., J. Bacteriol. 169:4923, 1987. The bacteria can be collected by centrifugation, and then subjected to an extraction protocol to isolate crude LPS. The crude LPS can be purified through known methods such as by polymyxin B affinity chromatography. Purified LPS can by lyophilized and stored for future use.

Portions of R. galegae LPS such as the lipid A portion are useful in the invention. Intact LPS can be fractionated into subcomponents by any suitable method. For example, the lipid A portion of R. galegae LPS can be obtained by subjecting the intact LPS to hydrolysis in 1% acetic acid for 1 h at 100° C. and collecting the resulting precipitate.

### Adjuvant Compositions and Vaccines

The R. galegae LPS or the lipid A portion thereof can be included in the adjuvant and vaccine compositions of the present invention. In addition to the R. galegae LPS or the lipid A portion thereof, the adjuvant compositions of the invention can further include an excipient, and the vaccine compositions can further include an excipient and at least one antigen. Any excipient suitable for use in the adjuvant and vaccine compositions of the invention might be used. For example, an oil might be used. Examples of oils include any suitable long-chain alkane, alkene or alkyne, or an acid, alcohol, ether or ester derivative thereof.

To avoid a local reaction, metabolizable, non-toxic, oils such as vegetable oils and animal oils are preferred. Vegetable oils include those obtainable from nuts, seeds, or grains, such as peanut oil, soybean oil, coconut oil, olive oil, safflower oil, cottonseed oil, sunflower seed oil, sesame seed oil, and corn oil. Animal oils (which might include fats) include those from mammals (cows, pigs, horses, etc.) and those from fish (e.g., cod liver oil, shark liver oil, whale oil, squalene, and squalane).

In certain variations of the invention, the excipient might also include a vitamin component, an emulsifying agent, and an aqueous solution component. The vitamin component may include one or more of Vitamin A, Vitamin D3 and Vitamin E. Examples of emulsifying agents include naturally derived materials such as gums from trees, vegetable proteins, sugar-based polymers such as alginates and cellulose, oxypolymers or polymers having a hydroxide or other hydrophilic substituent on the carbon backbone having surfactant activity (e.g., povidone, polyvinyl alcohol, and glycol ether-based mono- and poly-functional compounds), long chain fatty-acid-derived compounds, soaps, anionic non-soap detergents, nonionic detergents, long chain sulfoxides, ampholytic detergents, and zwitterionic detergents. The aqueous solution component can include water or any suitable water-containing liquid such as normal saline or a buffered solution such as buffered saline (e.g., phosphate-buffered saline).

The vaccine compositions of the invention additionally include at least one antigen. The antigen can be any substance which can elicit an immune response when administered to an animal subject, e.g., a peptide, a protein, a nucleic acid, a metal, a carbohydrate, a drug, etc. The antigen can be dissolved or dispersed in a carrier such as water or a buffered salt solution. In some embodiments of the invention, the vaccine composition is prepared by mixing an antigen component with the adjuvant composition prior to use, e.g., just prior to administration to an animal subject.

If desired, further adjuvants or other biologically active or inert substances can be added to the adjuvant and vaccine compositions of the present invention.

The amount of each component included in any given adjuvant or vaccine composition of the invention might vary considerably. As an example, an oil might be included between about 1-99%, 5-95%, 10-90%, 20-80%, 30-70%, 40-60%, 50%, 40%, 30%, 20%, 10%, or 5% (vol:vol) of the adjuvant or vaccine composition. A vitamin component might be included between about 1-99%, 5-95%, 10-90%, 20-80%, 30-70%, 40-60%, 50%, 40%, 30%, 20%, 10%, 5%, 4%, 3%, 2%, or 1% (vol:vol) of the adjuvant or vaccine composition. An emulsifying agents might be included between about 1-10%, 10%, 5%, 4%, 3%, 2%, 1%, 0.5%, or 0.1% (vol:vol) of the adjuvant or vaccine composition. An optimal amount for each component can be determined empirically for any given composition and application.

To augment an immune response in an animal subject (e.g., a mammal such as a cat, dog, horse, cow, rat, mouse, pig, sheep, monkey, ape, or human being), the adjuvant composition is administered to the subject at the same site that the antigen is administered. In the case of an antigen-containing vaccine composition, both the antigen and adjuvant components are administered simultaneously. In case of separate antigen and adjuvant compositions, the antigen composition and adjuvant compositions might be administered at different times to the same site. After a primary immunization, subsequent (booster) immunizations may be given to an animal subject to enhance the initial immune response. The compositions of the invention are preferably administered to an animal subject in an effective amount, that is, an amount capable of producing a desirable result in a treated subject (e.g., enhancing an antigen-specific immune response without inducing a significant inflammatory response).

### EXAMPLES

The present invention is further illustrated by the following specific examples. The examples are provided for illustration only and are not to be construed as limiting the scope or content of the invention in any way.

### Example 1

### Purification and Characterization of LPS from R. galegae

### In Vitro Complement Cleavage Assays

LPS from two nitrogen-fixing soil bacteria, R. galegae and R. sin-1, were tested for their ability to activate C′ in a simple in vitro assay. The C′ activating capacity of these bacteria was compared with that of E. coli LPS (O55:B4) and the LPS of S. typhimurium and S. minnesota R959 to offer a frame of reference. Experiments indicated that both R. galegae and R. sin-1LPS were about twice as effective as E. coli LPS at inducing C′ activation, with 50% activation doses (ED₅₀) of 6.25 and 9.3 μg/ml for the rhizobial LPS, respectively, versus 14.2 μg/ml for E. coli, and more than three times as effective as the two Salmonella LPS, that had a ED₅₀ of 33.7 and 33.3 μg/ml. Cleavage of C′ was dose dependent for all the LPS preparations once the threshold dose was achieved. R. galegae, E. coli, and S. minnesota lipid A all cleaved complement in a dose-dependent fashion. R. galegae lipid A had an ED₅₀ of 0.5 μg/ml, which was 7-fold lower than E. coli and 16-fold lower than S. minnesota lipid A, respectively.

### Materials and Methods

Bacterial Strains and Growth Conditions.: Rhizobium sp. (Sesbania) Sin-1 (R. Sin-1) was originally isolated from the root nodules of Sesbania aculeata, a tropical legume grown in Tamil Nadu, India. R. galegae HAMBI 540 is the type strain for this species. R. galegae is the symbiotic nitrogen fixing bacteria associated with the legumes Galega orientalis and Galega officinalis (Rana, D. and H. B. Krishnan, FEMS Microbiol. Lett. 134:19-25, 1995). Both bacterial strains were grown by fermentation in yeast extract mannitol (YEM) medium.

Isolation and Purification of LPS. Crude LPS was isolated by hot phenol/water extraction (see, Westphal, O. and K. Jann, Meth Enzymol. 5:83-91, 1965). The crude LPS present in the aqueous phase was further treated with RNase, DNase, and proteinase K, and then dialyzed and lyophilized. The LPS was then purified using polymyxin B affinity chromatography as previously described (Forsberg, L. S. and R. W. Carlson, J. Biol Chem., 273:2747-2757, 1998; Jeyaretnam et al., J. Biol Chem., 277:41802-41810, 2002; and Ridley et al., Glycobiology, 10:1013-1023, 2000). In the case of R. Sin-1, the LPS was separated into its “smooth” (LPS-containing O-antigen polysaccharide) and “rough” (LPS-lacking O-antigen polysaccharide) forms by gel filtration chromatography in the presence of deoxycholate. The “rough” LPS was used in the work described herein. Lipid A was prepared by mild acid hydrolysis of the LPS in 1% acetic acid at 100° C. for 1 h followed by centrifugation to collect the precipitated lipid.

Assay Chemicals and Reagents: The LPS from E. coli 055:B5, S. minnesota R959, and S. typhimurium, and lipid A from E. coli K12 D31m4and S. minnesota R595 were purchased from List Biologicals (Campbell, Calif.). The LPS and lipid A were prepared as a 1 mg/ml suspension in Dulbecco's phosphate buffered saline (PBS) by sonicating three times for thirty seconds at 75% power. Working samples were stored frozen at −80° C. until use. The suspension was vortexed extensively when thawed. The rhizobial LPS preparations were prepared at 1 mg/ml in PBS by sonication just before use. Lipid A from R. galegae was prepared in PBS at 500 ug/ml in PBS as described for the rhizobial LPS.

Guinea pig complement was purchased from ICN (Irvine, Calif.) and stored at −80° C. until use. Each vial was reconstituted in PBS, and split into sub-portions for the experiments in small volumes. These vials were stored at −80° C. and thawed only once more. Sheep erythrocytes were purchased from ICN and used within 14 days of receipt. Gelatin veronal buffer, with and without EDTA (GVB and GVE), were purchased from Sigma (St. Louis, Mo). All other materials were of the highest quality available.

Complement activity assay: A sheep erythrocyte suspension (ICN) was washed three times in PBS, and suspended at 3% (v/v) in GVB. All samples were tested in quadruplicate wells of round bottom 96 well plates at a volume of 100 μl/well of sample material. The following were used as controls: GVB with complement diluted 1:30 or 1:100 (dependent on the lot used), but no LPS added (C′ lysis control), GVB with each dilution of the LPS preparations tested, but no complement (direct LPS lysis control), GVB buffer only (erythrocyte stability control), and deionized water only (total hemoglobin, maximum color control). To each well 100 μl of 3% erythrocyte suspension was added at room temperature. The plates were incubated for one hour at 37° C. The plates were mixed gently by rubbing plate bottoms with knuckle every 10 min. Following incubation, the plates were centrifuged for 10 min at 1877×g. 100 μl of supernatant from each well was transferred to wells of a flat bottom 96 well plate. The optical density of the samples was read at 570 nm using an ELISA plate reader. Background (GVB only) was subtracted from all of the other readings. The percent of control lysis, relative to the GVB with complement, but no LPS control, was calculated for each sample using the average of the four replicates.

LPS and lipid A interaction with C′: A two-fold serial dilution of each LPS preparation was made in GVB over a range of 100 μg/ml to 0.2 μg/ml in a final volume of 500 μl in 1.5 ml micro-centrifuge tubes. The tubes were chilled to 4° C. in a water-ice bath, and cold guinea pig complement was added to each tube to a dilution of 1:100. Tubes were incubated for 18 hours at 4° C. These tubes were rocked about every 4 hours during the incubation period to mix the contents. After 18 hours LPS tubes were removed from 4° C., and placed by concentration in quadruplicate wells of round bottom 96 well plates at a volume of 100 μl/well for assessment of the remaining C′. The assessments were repeated three times. Lipid A was tested as above, except that the concentration range tested was 50 μg/ml to 0.02 μg/ml.

Data analysis: The fraction of C′ remaining after the 18 hour incubation was expressed graphically against the log of concentration for each preparation of LPS with the error bars representing the SEM. The graphs were used to evaluate pairings for assessment of differences in C′ cleavage. The differences in the percentage of C′ remaining were evaluated using two-way ANOVA in Prism (Graph Pad Software, San Diego, Calif.). Significance was accepted at p<0.05, and trends at p<0.10. Further, the ED₅₀ for each the cleavage of C′ for each LPS was calculated using the routine within Prism.

### Results

A comparison of C′ cleavage by E. coli and rhizobial LPS: E. coli O55:B5 LPS was compared with R. sin-1 and R. galegae LPS over a 3-log concentration range. R. sin-1 induced significantly more (p<0.01) C′ cleavage than E. coli LPS at all concentrations in excess of 6.25 μg/ml. Both LPS preparations exhibited dose-dependent cleavage of C′ and induced significant C′ cleavage at all concentrations tested.

R. galegae LPS cleaved C′ significantly better than did E. coli LPS at all concentrations above 10 μg/ml. R. galegae LPS also demonstrated significantly increased C′ cleavage at 1.5 μg/m relative to E. coli LPS. Again, both LPSs demonstrated dose-dependent cleavage of C′ over the range tested with R. galegae LPS inducing significant cleavage of C′ from 1.5 μg/ml, and E. coli LPS inducing significant cleavage of C′ at all concentrations tested.

A comparison of C′ cleavage by S. typhimurium and S. minesota R595 LPS, and rhizobial LPS: S. typhimurium LPS was compared with R. sin-1 and R. galegae LPS over a 3-log concentration range. R. sin-1 LPS induced significantly more (p<0.05) C′ cleavage than S. typhimurium LPS at all concentrations in excess of 3.125 μg/ml, with all concentrations above 6.25 μg/ml highly significant (p<0.01). S. typhimurium LPS induced significant cleavage of C′ at all concentrations tested, but was dose dependent from 25 to 100 μg/ml. In contrast, R. sin-1 LPS significantly cleaved C′ at all concentrations tested in a dose-dependent manner.

R. galegae LPS cleaved C′ significantly (p<0.01) better than did S. typhimurium LPS at all concentrations above 1 μg/ml. Again, S. typhimurium LPS induced significant cleavage of C′ at all concentrations tested, but was dose-dependent only from 25 to 100 μg/ml. In contrast, R. galegae LPS cleaved significant amounts of C′ at all concentrations tested and in a dose-dependent manner.

S. minesota LPS was compared with R. sin-1 and R. galegae LPS over a 3-log concentration range. R. sin-1 LPS induced significantly more (p<0.01) C′ cleavage than S. minesota LPS at all concentrations of 1.5 μg/ml and higher. R. galegae LPS cleaved C′ significantly (p<0.01) better than S. typhimurium LPS at all concentrations above 0.6 μg/ml. S. minesota LPS significantly induced C′ cleavage at all concentrations tested, and the cleavage of C′ was dose-dependent from 25 to 100 μg/ml. In these experiments, both R. sin-1 and R. galegae LPS cleaved C′ in a dose-dependent manner across the entire range tested.

Cleavage of C′ by R. galegae, E. coli, and S. minnesota lipid A: R. galegae lipid A was compared to commercial preparations of enteric lipid A in a separate set of experiments. E. coli and S. minnesota lipid A were compared with R. galegae lipid A over a 3-log concentration range. R. galegae lipid A induced significantly more (p<0.01) C′ cleavage than E. coli lipid A at all concentrations in excess of 0.5 μg/ml. Both lipid A preparations exhibited dose-dependent cleavage of C′ and induced significant C′ cleavage at all concentrations tested.

R. galegae LPS cleaved C′ significantly better than S. minnesota lipid A at all concentrations above 0.02 μg/ml. Again, both S. minnesota and R. galegae lipid A demonstrated dose-dependent cleavage of C′ over the range tested, inducing significant cleavage of C′ at all concentrations tested.

Comparison of the ED₅₀ for the LPS tested: Composite curves for all LPS trials were generated. There was a clear progression of ED₅₀ observed in these experiments. Both rhizobial LPS preparations had ED₅₀ below 10 μg/ml (6.6 μg/ml for R. galegae and 9.3 μg/ml for R. sin-1). E. coli LPS was intermediate with an ED₅₀ of 14.2 μg/ml, and both Salmonella LPS with ED₅₀ of about 33 μg/ml. R. galegae lipid A had an ED₅₀ of 0.5 μg/ml, compared with 3.3 μg/ml and 7.8 μg/ml for E. coli and S. minnesota lipid A, respectively. The relationship of LPS and lipid A C′ activation for R. galegae was about 14but only about 4-fold for E. coli or S. minnesota R595.

### Example 2

### Other In Vitro Results

R. galegae LPS did not induce TNF-alpha production in an in vitro culture of monocytes. In bovine cells, R. galegae LPS induced increased radical oxygen (but not nitrogen) production, B cell proliferation, increased CD25 expression, and increased particle uptake by phagocytes.

### Example 3

### Animal Experiments

Materials and Methods: LPS was prepared or obtained as described in Example 1. Vaccine compositions were prepared by emulsion of 10% olive oil containing 100 IU Vitamin E, 50 IU Vitamin D3, 10 μg of Vitamin A per ml in a PBS base containing antigen (3000 μg/ml of bovine serum albumin [BSA]), and 100 μg/ml LPS. The emulsion was prepared by passing the mixture 40 times through a 30 gauge emulsion needle between a pair of syringes. Primary and booster vaccinations were given as 1 ml per rabbit (about 2 Kg) in 2 IM sites on the neck or 4 IM sites in the thighs, respectively. Animals were separated into 4 groups based on treatment: adjuvant only, BSA-vaccine with R. galegae LPS, BSA-vaccine with E. coli LPS, and BSA-vaccine with Alhydrogel. Anti-BSA antibody titer was quantified at various periods after administration.

Results: After vaccination of rabbits with an R. galegae LPS-containing vaccine composition, all animals showed primary and secondary antibody responses comparable to that induced by a commercial adjuvant (Alhydrogel). The primary response reached about 3 logs higher than background for all groups, and the secondary response, peaking 1 week after boost, was better than 4.5 logs over background.

Fecal antibody was also assessed. The antibody response in serum was about 1000 times greater than that measured in feces (a mucosal antibody response). Five of six animals vaccinated with the R. galegae LPS-containing vaccine showed significant mucosal antibody production in the second week following vaccine booster. In contrast, only one in six animals receiving the E. coli LPS-containing vaccine and one in six animals receiving the commercial adjuvant-containing vaccine had a significant mucosal antibody response at the same time point. The peak of mucosal antibody response followed the peak serum antibody response by one week. The mucosal response reach about 1-2 logs above background at peak. The highest mucosal responses were 3 logs lower than serum responses in the animals tested. The number of lymph nodes draining the injection sites that were large enough to recover was fewer in animals receiving the R. galegae LPS-containing vaccine compared to animals receiving the E. coli LPS-containing vaccine.

Importantly, the R. galegae LPS-containing vaccine did not induce fever or other physiological signs of inflammation during the 48 hours following IM delivery. Further, no rejection site reactions were observed in necropsy 5 weeks following primary and 3 weeks following secondary (different sites) vaccination with the R. galegae LPS-containing vaccine or an equivalent adjuvant composition lacking antigen.

In a second similar rabbit experiment, test animals (about 3 Kg) were separated into 4 groups based on treatment: adjuvant only, adjuvant with antigen (but no LPS), BSA-vaccine with R. galegae LPS, BSA-vaccine with E. coli LPS, and BSA-vaccine with Alhydrogel. The adjuvant with antigen (but no LPS), BSA-vaccine with R. galegae LPS, BSA-vaccine with E. coli LPS were similar at inducing systemic antibody production (about 5 logs increase by 4 weeks after vaccination, and mostly IgG). The adjuvant with antigen (but no LPS) and BSA-vaccine with R. galegae LPS were similar in that both induced earlier, higher and more sustained mucosal responses than commercial adjuvant.

The level of fecal antibody was significantly higher on week 4 for the R. galegae LPS-containing vaccine composition compared to all others in first experiment, and was higher than E. coli LPS-containing vaccine or AlOH-containing vaccine at week 4 in the second experiment. In the second experiment, the adjuvant with antigen (but no LPS) induced a significantly higher titer than all others on weeks 2, 3 and 5, but the titer was not significantly greater than that induced by the BSA-vaccine with R. galegae LPS on week 4. Both the adjuvant with antigen (but no LPS) and the BSA-vaccine with R. galegae LPS induced significantly higher mucosal responses than did the BSA-vaccine with E. coli LPS or the BSA-vaccine with Alhydrogel at week 4. The BSA-vaccine with R. galegae LPS damped the mucosal antibody production effect observed using the adjuvant with antigen (but no LPS) composition, but this was less than the damping observed with the BSA-vaccine with E. coli LPS or the BSA-vaccine with Alhydrogel (which induced similar levels of mucosal antibody production).

Necropsy findings in both the first and second rabbit experiments indicated that no systemic inflammatory side-effects were associated with the vaccines tested and no site reactions were induced. In the first experiment, no differences in spleen or lymph nodes were observed among the vaccine groups.

### Example 4

### Additional Animal Experiments

Vaccine experiments were also performed in mice. R. galegae LPS without oil was additionally tested (at 20, 40 and 80 μg/kg weight). It appeared to have dose-dependent activity, inducing between a 5- and 40-fold increase in BSA antibody titer in serum (much less than the 100,000 fold increase observed with the R. galegae LPS with olive oil combination). It also induced an apparent lymphocyte recall proliferation response to BSA and induced anti-BSA antibody secretion into fecal mucus.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An adjuvant composition comprising an excipient and a purified R. galegae LPS.

2. The adjuvant composition of claim 1, wherein the excipient comprises an oil.

3. The adjuvant composition of claim 2, wherein the oil is a metabolizable oil.

4. The adjuvant composition of claim 3, wherein the metabolizable oil is olive oil.

5. The adjuvant composition of claim 1, wherein the excipient comprises a vitamin selected from the group consisting of vitamin A, vitamin D3, and vitamin E.

6. The adjuvant composition of claim 5, wherein the excipient further comprises a metabolizable oil.

7. An adjuvant composition comprising an excipient and a purified lipid A portion of an R. galegae LPS.

8. The adjuvant composition of claim 7, wherein the excipient comprises an oil.

9. The adjuvant composition of claim 8, wherein the oil is a metabolizable oil.

10. The adjuvant composition of claim 9, wherein the metabolizable oil is olive oil.

11. The adjuvant composition of claim 7, wherein the excipient comprises a vitamin selected from the group consisting of vitamin A, vitamin D3, and vitamin E.

12. The adjuvant composition of claim 11, wherein the excipient further comprises a metabolizable oil.

13. A vaccine composition comprising an adjuvant selected from the group consisting of a purified R. galegae LPS and a purified lipid A from R. galegae LPS; and an antigen.

14. The vaccine composition of claim 13, further comprising an excipient.

15. The vaccine composition of claim 14, wherein the excipient comprises an oil.

16. The vaccine composition of claim 15, wherein the oil is a metabolizable oil.

17. The vaccine composition of claim 16, wherein the metabolizable oil is olive oil.

18. The vaccine composition of claim 14, wherein the excipient comprises a vitamin selected from the group consisting of vitamin A, vitamin D3, and vitamin E.

19. The vaccine composition of claim 18, wherein the excipient further comprises a metabolizable oil.

20. A method of modulating an immune response in an animal, the method comprising administering to the animal an effective amount of a vaccine comprising an adjuvant comprising a purified R. galegae LPS, and an antigen.

21. The method of claim 20, wherein the immune response is a mucosal antibody production response.

22. The method of claim 20, wherein the immune response is a serum antibody production response.

23. A method of modulating an immune response in an animal, the method comprising administering to the animal an effective amount of a vaccine comprising an adjuvant comprising a purified lipid A from R. galegae LPS, and an antigen.

24. The method of claim 23, wherein the immune response is a mucosal antibody production response.

25. The method of claim 23, wherein the immune response is a serum antibody production response.
